(19) United States
(12) Patent Application Publication
(10) Pub. No.: US 2026/0115356 A1
(43) Pub. Date: Apr. 30, 2026

(54) ANGIOGENESIS STIMULATOR COPPER/MAGNESIUM HYDROXIDE HYBRID NANOPARTICLE

(71) Applicant: Marquette University, Milwaukee, WI (US)

(72) Inventors: Lobat Tayebi, Milwaukee, WI (US); Meisam Omidi, Milwaukee, WI (US)

(73) Assignee: Marquette University, Milwaukee, WI (US)

(21) Appl. No.: 19/473,730

(22) PCT Filed: Apr. 26, 2024
(86) PCT No.: PCT/US2024/026567

§ 371 Date: Oct. 8, 2025

Related U.S. Application Data
(60) Provisional application No. 63/499,006, filed on Apr. 28, 2023.

(51) Int. Cl. A61L 27/30; A61L 27/18

(52) U.S. Cl. CPC A61L 27/306; A61L 27/18; A61L 2400/12

(57) ABSTRACT

A scaffold for bone-tissue engineering includes a mesh and a coating on the mesh. The mesh includes a plurality of offset layers of filaments of polyester. The coating covers the mesh and includes magnesium hydroxide/copper oxide nanoparticles suspended in gelatin. A method of constructing the scaffold comprises combining magnesium hydroxide/copper oxide nanoparticles with a gelatin solution and coating a polyester mesh with the combined magnesium hydroxide/copper oxide nanoparticle and gelatin solution.

### CROSS-REFERENCE TO RELATED APPLICATIONS

The present application claims priority of U.S. Provisional Patent Application No. 63/499,006, filed on Apr. 28, 2023, the contents of which is incorporated by reference herein in its entirety.

### STATEMENT REGARDING FEDERALLY SPONSORED RESEARCH OR DEVELOPMENT

This invention was made with government support under 1R56 DE029191-01A1 awarded by the National Institute of Dental & Craniofacial Research of the National Institutes of Health. The government has certain rights in the invention.

### BACKGROUND

Bone tissue engineering to repair large bone defects presents a significant clinical challenge as bone regeneration is a complex, multistep physiological process. Although tissue-engineered scaffolds based on novel biomaterials can repair bone defects, not all scaffolds can simultaneously address issues of biocompatibility, biodegradability, mechanical properties, and high osteoconductivity. Biopolymers and bioceramics are the most commonly used as biomaterials for bone scaffolds. Biodegradable polyesters like polycaprolactone (PCL) have been used for drug delivery, surgical treatments, and implantable medical devices because of their unique characteristics including biodegradability, processability, and controlled strength. However, acidic byproducts during biopolymer degradation can decrease the environmental pH and induce an inflammatory response.

Nanomaterials have been used as an alternative approach to improve biomaterial properties, especially metallic nanostructures due to their unique biological, chemical, physical, and mechanical properties. The osteogenic and angiogenic properties of magnesium (Mg) and copper (Cu) are known. It is also known that Mg can buffer the produced acidic byproduct and suppress inflammatory response during biopolymer degradation. Copper can improve healing and maintaining bone volume, can upregulate vascular endothelial growth factor (VEGF) gene expression, and induce osteogenic differentiation in mesenchymal stem cells (MSCs). Although Cu prevents bone infections around implanted scaffolds, high local concentrations of Cu can be toxic.

U.S. Pat. No. 11,026,794, entitled, “Reinforced Bone Scaffold,” discloses a scaffold for use in bone tissue engineering and methods of preparation of such scaffolds. The contents of this patent is hereby incorporated by reference in its entirety. US Patent Application Publication No. 2021/0400984, entitled, “Antimicrobial Adhesive Composition with Copper Nanoparticles for Dentures,” discloses a denture adhesive composition with copper nanoparticles (CuNPs). The contents of this patent application is hereby incorporated by reference in its entirety.

Yang, et al. “Cu-releasing bioactive glass/polycaprolactone coating on Mg with antibacterial and anticorrosive properties for bone tissue engineering” discloses bioactive glass nanoparticles containing copper (C-BGNs) introduced into polycaprolactone (PCL) coating systems to improve the bioactivity, antibacterial properties, and corrosion resistance of magnesium matrices under physiological conditions. The Cu-BGN/PCL composite coatings imparted antibacterial and anticorrosive properties to magnesium-based biomaterials for clinical applications. The contents of this paper is hereby incorporated by reference in its entirety.

Suryavanshi, et al. “Magnesium oxide nanoparticle-loaded polycaprolactone composite electrospun fiber scaffolds for bone-soft tissue engineering applications: in-vitro and in-vivo evaluation” discloses an assessment of magnesium oxide nanoparticle (MgO NP)-loaded electrospun polycaprolactone (PCL) polymer composites as a bone/soft tissue engineering scaffold. The contents of this paper is hereby incorporated by reference in its entirety.

### BRIEF DISCLOSURE

A polyester based 3D printed scaffold coated with Mg(OH)2/CuO hybrid nanostructure (MCNs) and gelatin for effective bone regeneration.

An example of a scaffold for bone-tissue engineering includes a mesh and a coating on the mesh. The mesh includes a plurality of offset layers of filaments of polyester. The coating covers the mesh and includes magnesium hydroxide/copper oxide nanoparticles suspended in gelatin.

The polyester of the mesh may be polycaprolactone (PCL). The magnesium-copper nanoparticles may have a concentration of 0-5 g copper acetate per 75 mL of 2M magnesium chloride solution. The magnesium-copper nanoparticles may have a concentration of 1-4 g copper acetate per 75 mL of 2M magnesium chloride solution. The magnesium-copper nanoparticles may have a concentration of 1.2-3.6 g copper acetate per 75 mL of 2M magnesium chloride solution. The magnesium-copper nanoparticles may have a concentration of 1.6-2.4 g copper acetate per 75 mL of 2M magnesium chloride solution. The mesh may have a porosity of up to 1 mm×1 mm or may have a porosity of 400 μm×400 μm. The coating may be up to 2 μm thick, or may be between 1 μm-2 μm thick.

In examples of the scaffold, the copper oxide includes at least copper(II) oxide. The copper oxide may include both copper(I) oxide and copper(II) oxide. A ratio of copper(I) oxide to copper(II) oxide may be less than 0.42. The ratio of copper(I) oxide to copper(II) oxide may be less than 0.32. The magnesium hydroxide/copper oxide nanoparticles may have an average particle size of 150 nm or less. The magnesium hydroxide/copper oxide nanoparticles may have an average particle size of about 120 nm. The magnesium hydroxide/copper oxide nanoparticles may be present in the gelatin at a concentration of 40 mg-60 mg of magnesium hydroxide/copper oxide nanoparticles to 1 mL gelatin. The scaffold may have an average pore size of between 351 μm-505 μm. The scaffold may be configured to maintain an invivo localized pH greater than 3.

An example of a method of constructing a scaffold for bone-tissue engineering includes 3D printing a mesh including a plurality of offset layers of filaments of polyester. Magnesium hydroxide/copper oxide nanoparticles are prepared by combining 2M solution of magnesium chloride with copper acetate. The magnesium hydroxide/copper oxide nanoparticles are combined with a gelatin solution at a concentration of 40 mg/mL to 60 mg/mL. Amino-functional groups introduced to the mesh by immersing the mesh in a solution of 1,6 hexanediamine in isopropanol. The mesh is soaked in the combined magnesium hydroxide/copper oxide nanoparticle and gelatin solution.

### BRIEF DESCRIPTION OF THE DRAWINGS

FIG. 1 is a diagram depicting the process of constructing the scaffold as described herein.

FIG. 2 is a diagram of an example scaffold.

FIG. 3A is a photograph image of a PCL scaffold mesh.

FIG. 3B is a photograph image of a PCL/gelatin/MCN-2 scaffold.

FIG. 4A are SEM micrographs of MCN samples.

FIG. 4B are TEM micrographs of MCN samples.

FIG. 4C is a graph of XRD patterns of MCN samples.

FIG. 4D is a graph of XPS spectra of MCN samples.

FIG. 5A are SEM micrographs at two scales of tested scaffolds.

FIG. 5B is a graph of water contact angles for the tested scaffolds.

FIG. 5C is a graph of mass loss over time of for the tested scaffolds.

FIG. 5D is a graph of pH changes over time for the tested scaffolds.

FIG. 6A is a graph of released Cu ions over time.

FIG. 6B is a graph of released Mg ions over time.

FIG. 6C are SEM micrographs of MCN-2 scaffold degradation over time.

FIGS. 7A-D are graphs representing osteogenesis activity.

FIGS. 8A and 8B are graphs representing angiogenesis activity.

FIGS. 9A and 9B are graphs representing in vivo test results.

FIG. 10 is an EDS spectrum of MCN-2.

### DETAILED DISCLOSURE

A new scaffold for bone tissue engineering is disclosed herein. The scaffold combines a polyester 3D printed mesh coated with gelatin and new magnesium hydroxide (Mg(OH)2)/copper oxide hybrid nanoparticles (MCNs). More specific examples include hybrid nanoparticles comprising magnesium hydroxide (Mg(OH)2)/cupric oxide (CuO). Laboratory observations conclude that the disclosed apparatus promotes angiogenesis and osteogenesis, improving patient healing and bone regeneration in treatment of defects including critically sized bone loss due to injury, disease, or genetics. Unless otherwise stated, use of the term “about” is to be interpreted generally to mean +/−20% of the values stated, recognizing that many values may represent an average value across a normal distribution of value results.

Polyesters including, but not limited to: polycaprolactone (PCL), polylactic acid (PLA), and polylactic acid-co-glycolic acid (PLGA) are attractive materials for use in constructing bone tissue engineering scaffolds. These materials exhibit proper and programmable degradation, good mechanical integrity, and low rigidity, resulting in manageable surgical handling. These materials are also usable with 3D printing techniques, are cost effective, exhibit sufficient strength and density, and are biodegradable. However, the biodegradation of polyesters produces acidic byproducts which counteract new tissue generation particularly new bone tissue, and the acidic byproducts invoke an inflammatory response by the body which further inhibits healing and tissue generation. The inventors have discovered that the combination of a polyester mesh with a coating of the MCNs disclosed herein create a self-regulating bone tissue engineering scaffold, which promotes, rather than inhibits, cell growth.

The MCNs are synthesized using a hydrothermal process. A 2M solution of magnesium chloride (MgCl₂), available from Sigma-Aldrich (USA), is provided in an exemplary amount of 75 mL. Copper acetate (Cu(OAc)₂), available from Sigma-Aldrich (USA), is added to the magnesium chloride in an amount to achieve an intended copper concentration in the MCN formulation. In examples, this is 0-5 g. In still further examples, this is between 1-4 g. In still further examples this is 1.2-3.6 grams or about 1.2-3.6 grams. The range may further be 1.6-2.4 grams or 2-3 grams. In still further examples, this may be 2.4 grams or about 2.4 grams. Copper acetate added in an amount of 1.6-2.4 grams to 75 mL of 2M magnesium chloride solution results in a concentration of copper in the range of 21-32 mg/mL or about 21-32 mg/mL. Subsequently, 20 mg NaOH and 50 mg L-Arginine, available from Sigma-Aldrich (USA), are gradually added to the solution under constant stirring. The solution is transferred to a hydrothermal vessel and purged with nitrogen for 5 minutes. The vessel is then sealed and transferred to an oven preheated to 160° C. and kept at that temperature for 8 hours. The vessel is then cooled to room temperature. The sample is then transferred to a beaker and washed with water and ethanol, respectively. Finally, the sample is dried in a vacuum oven at 50° C. for 24 hours. Experimental data referenced herein prepared according to the above with the following copper acetate content and reference names. Further examples of the disclosed nanoparticles will be recognized from these examples tested.

Copper/magnesium hydroxide hybrid
nanostructures (Mg(OH) ₂/CuO)

| Reference Name | Reference Copper Acetate content |
|---|---|
| Mg(OH)₂ | 0 g |
| MCN-1 | 1.2 g |
| MCN-2 | 2.4 g |
| MCN-3 | 3.6 g |

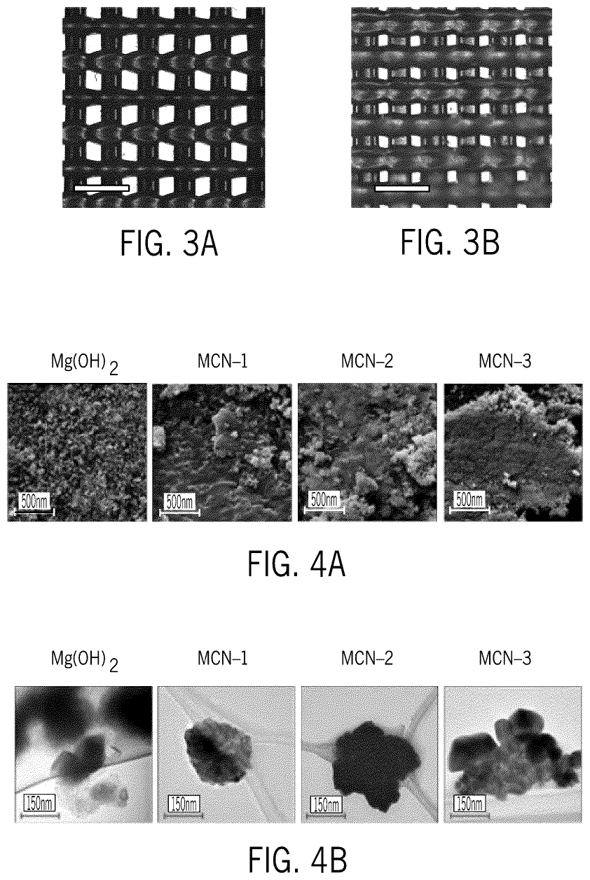

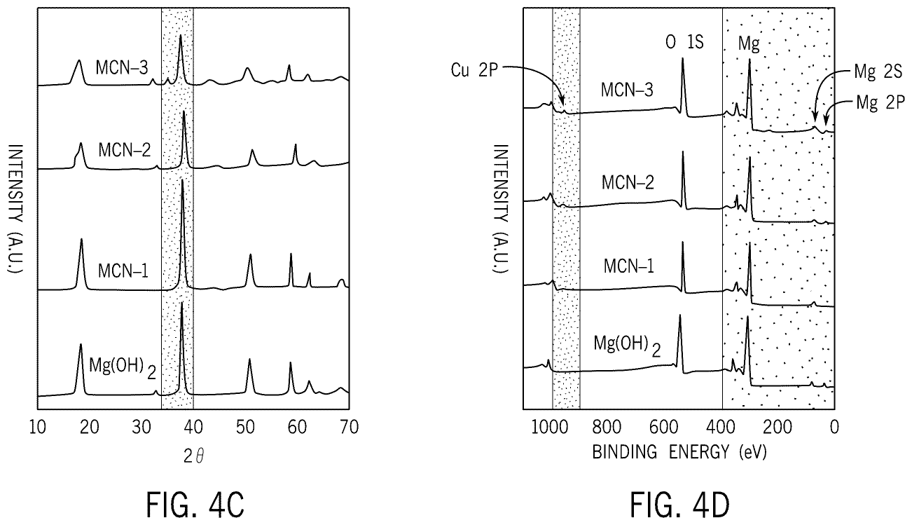

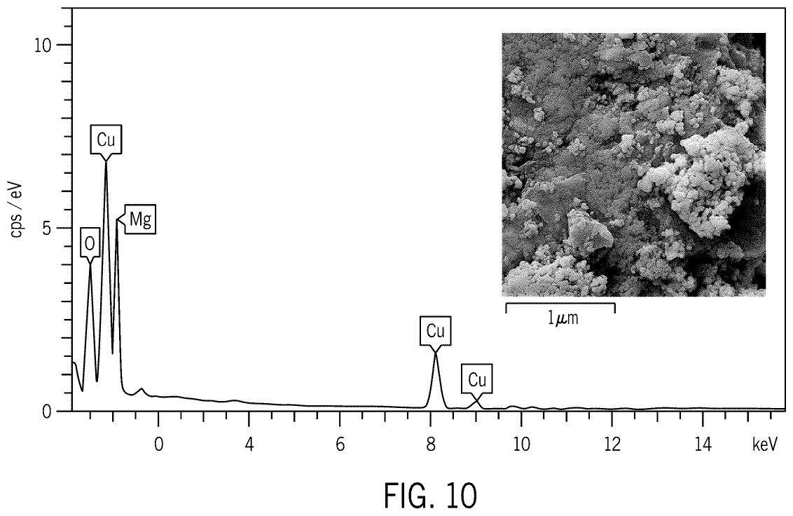

FIGS. 4A-D present investigative characterizations of MCN disclosed herein with varying copper concentrations. FIG. 4A presents SEM micrographs and FIG. 4B presents TEM micrographs of exemplary MCN formulations. Comparative observation of these images show that the morphology of the nanostructure changes with increased Copper content. A flake-like morphology with an average particle size of 110 nm can be observed for pure Mg(OH)₂ (e.g. no copper content). MCN-1, with the 1.2 g copper formulation above, exhibits the same morphology with a larger average particle size (150 nm). The size increment was observed for samples with higher copper content, for example MCN-2, with the 2.4 g copper formulation above, and MCN-3, with the 3.6 g copper formulation above. The agglomeration of the particles also significantly rose with increased copper content. Notable large clusters with small deposited particles are apparent in SEM (FIG. 3A) images of MCN-2 and MCN-3. FIG. 10 is a graph of EDX analysis showing the distribution of Cu in the MCN-2 hybrid nanostructure.

FIG. 4C presents graphical results of XRD analysis of the exemplary MCN formulations. The XRD analysis revealed that the introduction of copper oxides in the Mg(OH)2 base material resulted in changes in the crystallinity and lattice strain of the MCN samples. In the presence of copper, the reflection of (101) planes decreased. The lattice strain increased with higher copper content, suggesting distortions in the brucite structure caused by the substitution of Cu2+ for Mg2+. This distortion may be attributed to the larger ionic radius of Cu2+ compared to Mg2+, which could promote the Jahn-Teller effect. This structural change might be the main reason for diminishment in the intensity of reflections. Furthermore, previous reports have shown that the chance of forming the nanometric cluster increases during nanostructure fabrication in the presence of higher loads of Cu (>4.5%). This phenomenon might be another reason for reflections' pick diminishing. The crystallinity of CuO in nanoparticles may exert a significant influence on endothelial cell proliferation, migration, and tube formation. X-ray diffraction (XRD) analysis further revealed that MCN-2 exhibited a monoclinic crystal structure with smaller lattice parameters than the other samples tested. As higher crystallinity is known to promote angiogenesis, MCN-2 may be deemed an optimized condition for this purpose.

FIG. 4D presents graphical results of XPS survey spectrum of the exemplary MCN formulations. These graphs show the Mg 2p and Mg 2s peaks for all samples and the Cu 2p_{1/2} and Cu 2p_{3/2} peaks for samples with copper (MCN-1, MCN-2, and MCN-3). Also, the XPS spectra of MCN-1, MCN-2, and MCN-3 samples, could be deconvoluted into the two peaks (932 eV and 934 eV) that represent Cu⁺ and Cu²⁺, respectively. These peaks confirm the presence of different states of copper oxides, Copper(I) oxide (Cu₂O) and Copper(II) oxide (CuO), the MCNs. X-ray photoelectron spectroscopy (XPS) analysis enables the determination of the binding energy of Cu 2p electrons, thus providing valuable information on the oxidation state of Cu in the hybrid nanostructure. The peak area associated with Cu2O/CuO ratio was found to be highest in MCN-1, suggesting a higher amount of Cu2O in this sample. This could be attributed to a more efficient heating process during the synthesis, leading to localized higher temperatures and reduction of CuO to Cu2O. The peak area associated with the Cu2O/CuO ratio was found to be lowest in MCN-2, suggesting a higher amount of CuO in this sample. The oxidation state of copper in nanoparticles may exert a crucial influence on angiogenesis promotion. Specifically, the Cu(II) oxidation state can upregulate pro-angiogenic factors and stimulate the production of reactive oxygen species (ROS), thereby promoting angiogenesis. Based on our results, it can be inferred that MCN-2, characterized by the highest Cu(II) oxidation state among all samples, could be considered as an optimized condition.

Table 1 below presents a summary of the samples tested along with experimentally determined structural and functional characteristics of those samples.

TABLE 1

| | Copper | | | | |
|---|---|---|---|---|---|
| | Acetate | | Average | The | Cu2O/CuO |
| | content | | Particle | crystallite | (Cu(I)/Cu(II)) |
| | (per 75 mL | | Size | size (nm) | ratio from |
| Sample | Mg(OH)² | Morphology | (nm) | from XRD | XPS |
| Mg(OH)2 | 0.0 g | Flake-like | 160 | 97 | N/A |
| MCN-1 | 1.2 g | Flake-like | 150 | 97 | 0.42 |
| MCN-2 | 2.4 g | Agglomerated | 120 | 30 | 0.28 |
| | | with large | | | |
| | | clusters and | | | |
| | | small deposited | | | |
| | | particles | | | |
| MCN-3 | 3.6 g | Agglomerated | 210 | 31 | 0.32 |
| | | with notable | | | |
| | | large | | | |
| | | clusters | | | |
| | | and small | | | |
| | | deposited | | | |
| | | particles | | | |

Based upon the results of the XRD and XPS analysis MCN-2 is believed to be the most effective in promoting angiogenesis due to its higher crystallinity, smaller lattice parameters, and higher Cu(II) oxidation state. Cu(II) oxidation state can upregulate pro-angiogenic factors and stimulate the production of reactive oxygen species, which promote angiogenesis.

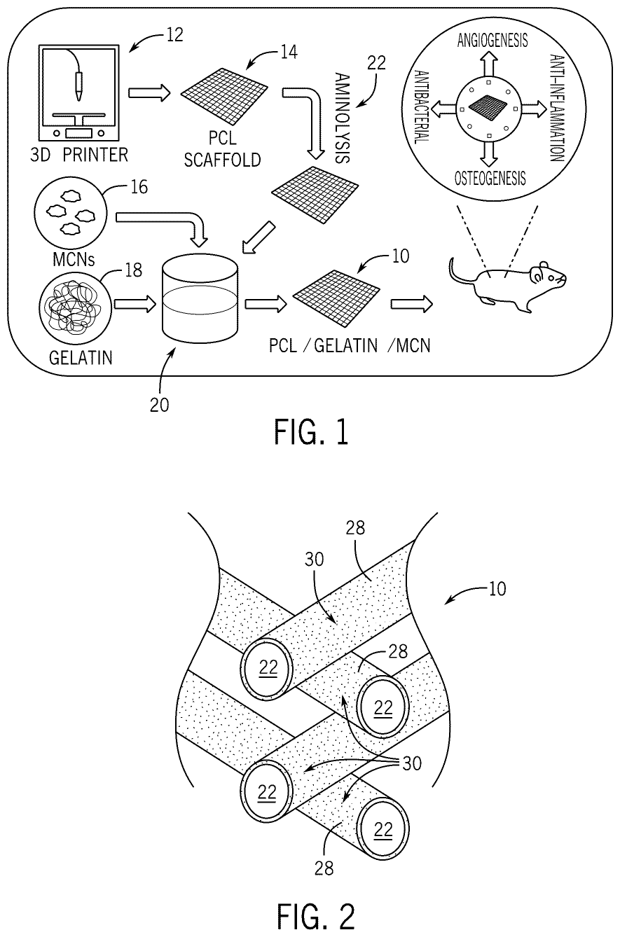

FIG. 1 is a diagram that depicts the process of the construction of the scaffold as will be described in further detail herein. The scaffold 10 is constructed with a 3D printer 12 to 3D print a mesh 14 of polyester, e.g. polycaprolactone (PCL) (45000 Mw: Sigma-Aldrich). The mesh 14 is exemplarily shown in the image of FIG. 3A. It is recognized that in clinical use and treatment, the areas of bone loss to be treated may be a three-dimensional space and irregular in shape. Therefore, in examples, a 3D design for the scaffold may be made with reference to computed tomography (CT) imagery of the patient and the treatment area to define the treatment area. From this defined treatment area, a computer-aided three-dimensional design of the mesh may be constructed and translated for 3D printing. In other examples, a standard volume (e.g. area and thickness) of the mesh is constructed and the mesh and/or resulting scaffold, shaped to the treatment area, for example by folding and cutting.

The PCL mesh 14 is exemplarily constructed of membrane layers constructed of 3D printed filaments. A needle possessing an internal diameter of 0.250 mm is utilized to fabricate membrane mesh with a porosity of 1 mm×1 mm. During the printing process, the chamber is maintained at a temperature of 180° C. and the platform is maintained at a temperature of 50° C. A pressure of 1.7 bar and a plotting speed of 20 mm/s are used. The mesh is constructed by sequentially printing layers as described above with each subsequent layer angled with respect to the previous layer. While the example described for experimental purposes herein offset each layer by 900 from the preceding layer, other angles, including but not limited to +/−30°, 45°, or 60° may be used. Similarly, while the example described herein for experimental purposes uses a mesh of four of the above-described layers, it will be recognized that any number of layers may be used to construct the thickness and/or volume of the intended scaffold.

The MCNs 16 are combined with gelatin 18 for coating the mesh. The MCNs in the selected Cu concentration as described above (e.g. 1.2-3.6 g or 1.6-2.4 g copper acetate to 75 mL 2M magnesium chloride solution), are added to gelatin (e.g. Type A, from porcine skin, Bioreagent grade, available from Elastin Products (USA)) solution (5% w/w in PBS) in an amount of 5 g or about 5 g MCN to 100 mL of gelatin solution to form a gelatin/MCN solution 20. In examples, the concentration of MCN in the gelatin solution may exemplarily range from 40 mg/mL to 60 mg/mL or from 45 mg/mL to 55 mg/mL.

The mesh 14 is prepared to accept the gelatin/MCN by introducing amino-functional groups through aminolysis at 22 using 1, 6-hexanediamine (available from Sigma-Aldrich (USA)). In an example of the procedure, the PCL mesh is washed three times with DI water and isopropanol. The clean mesh is immersed in 10% (w/w) solution of 1,6 hexanediamine in isopropanol at 40° C. for 30 minutes. The mesh is then washed with DI water and dried in a vacuum. The amino-functional groups on the mesh are chemically activated using glutaraldehyde (available from Sigma-Aldrich (USA)). The aminolyzed mesh is soaked in a 2% (w/w) glutaraldehyde solution for 24 hours at room temperature. Then the mesh is removed from the solution and washed five times with a large amount of DI water and dried in a vacuum.

The prepared mesh is then soaked in the gelatin/MCN solution 20 for 1 minute and incubated in 1-Ethyl-3-(3-dimethylaminopropyl) carbodiimide (EDC, Alfa Aesar (USA)) and NHydroxysuccinimide (NHS, Alfa Aesar (USA)) solution (6 mg/ml EDC and 0.75 mg/ml NHS in ethanol) for three hours at room temperature. Lastly the gelatin/MCN coated mesh is washed with DI water and lyophilized to produce the scaffold 10. In examples, this produces a scaffold 10 of the PCL printed mesh with an MCN/gelatin coating of about 1-2 μm thickness. This is diagrammatically depicted in FIG. 2. Filaments 22 of PCL each form stacked layers 24 of the mesh 26. The coating 28 surrounds each of the filaments 22 and is composed of gelatin with the MCN 30 distributed therein. FIG. 3B is an exemplary image of the scaffold 10 of a PCL mesh with the gelatin MCN-2 coating

In clinical use, the disclosed scaffold of a polyester mesh with the MCN/gelatin coating promotes angiogenesis and osteogenesis. In vivo, the biodegradation of the polyester mesh and the coating releases of polyester (or polyester decomposition byproducts), magnesium, and copper which regulate each other to achieve localized conditions to promote angiogenesis and osteogenesis for the healing of critically sized bone losses. The biodegradation of polyester, for example PCL in the mesh, produces an acidic byproduct, which induces an inflammatory response, which in turn inhibits healing.

As PCL degrades, it breaks down into smaller molecules, and one of the byproducts is caproic acid, also known as hexanoic acid. PCL is made up of repeating units derived from F-caprolactone. The structure can be represented as: (—O—(CH2)5-CO)n-(—O—(CH₂)₅—CO)n-. During hydrolysis, water molecules break the ester bonds between the repeating units of PCL. The degradation of PCL into caproic acid can be generally represented as follows:
(—O—(CH₂)₅—CO)n+n H₂O→n HO—(CH₂)₅—COOH(—O—(CH₂)₅—CO)n+nH₂O→nHO—(CH₂)₅—COOH where, (—O—(CH₂)₅—CO)n(-O—(CH₂)₅—CO)n represents the PCL polymer, H₂O represents water, and HO—(CH₂)₅—COOHHO—(CH₂)₅—COOH is caproic acid.

The hydrolysis leads to the cleavage of the PCL polymer chain into smaller chains and ultimately into monomers, caproic acid. The acidity in the environment where PCL is degrading can increase as more caproic acid is produced.

The byproduct, caproic acid, contributes to the acidity of the local environment as the PCL degrades. As pH decreases in this localized area about the scaffold from this acidic byproduct, release of MCNs is increased. The copper ions from the MCN inhibit bacterial growth and induce angiogenesis, counteracting any impairment of the healing process due to the inflammatory response. The magnesium ions from the MCN buffer the acidic byproduct, increasing the pH. Increasing of the pH also lowers the inflammatory response and subsequently slows the release of copper and magnesium ions of the MCN. The copper induced angiogenesis and neutral pH in turn further the biodegradation of the polyester, forming self-regulating conditions that find an equilibrium that promotes healing, angiogenesis, and osteogenesis.

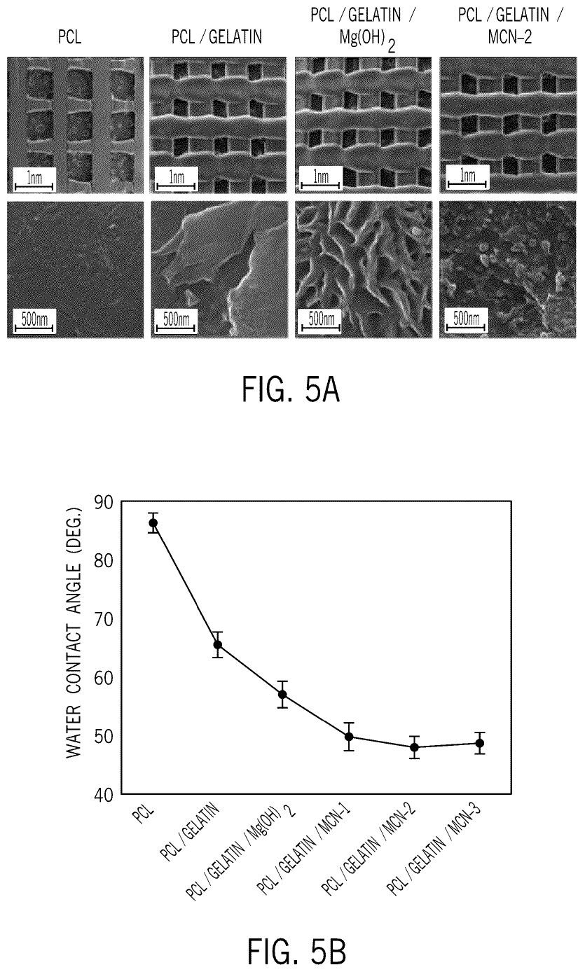

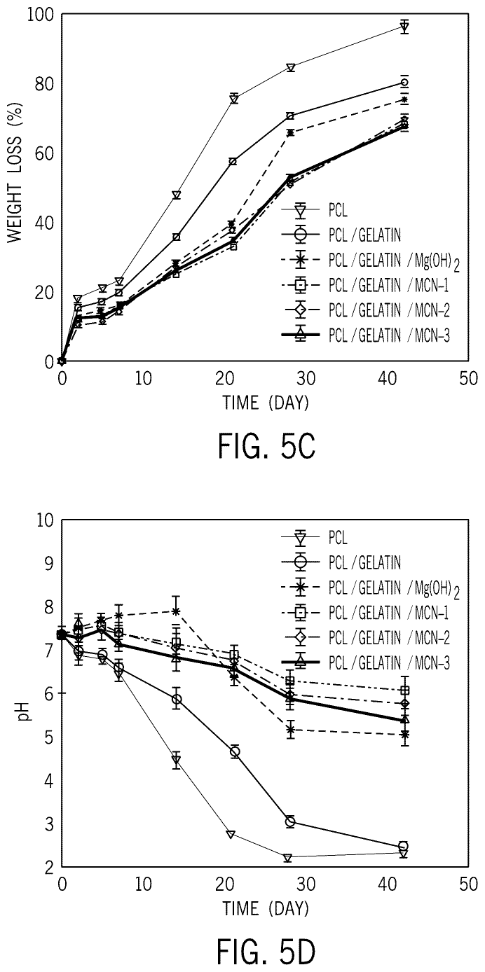

FIGS. 5A-5D present data of the physiochemical characterization of the scaffolds. FIG. 5A presents SEM micrographs of the PCL, PCL/gelatin, PCL/gelatin/Mg(OH)₂, and PCL/gelatin/MCN-2 scaffolds. These SEM results show that the porosity increased in the presence of MCN. The unconnected pore with an open-porous structure with an average pore size of 480±25 μm and 394±43 μm could be observed for PCL/gelatin/Mg(OH)₂ and PCL/gelatin/MCN-2 scaffolds, respectively. When taken together provide an average pore size of between 351 μm-505 μm. Mass transport phenomena is facilitated by an interconnected porous structure.

FIG. 5B is a graph of water contact angle and shows the decrease in water contact angle with the addition of gelatin, Mg(OH)₂, and MCN. The hydrophilicity indeed was evaluated using the surface wettability test. The addition of gelatin reduced the water contact angle from 88° to 62°, and the addition of MCN further reduced the water contact angle to 46°. Improving hydrophilic index can enhance mass transfer phenomena and cell-seeding in the scaffold. Table 2 below presents the water contact angles and standard deviation thereof for different tested samples.

TABLE 2
Water contact angles of samples

| Sample | water contact angle | SD |
|---|---|---|
| PCL | 86 | 1.72 |
| PCL/Gelatin | 63 | 2.52 |
| PCL/Gelatin/Mg(OH)2 | 54 | 2.7 |
| PCL/Gelatin/MCN-1 | 46 | 2.76 |
| PCL/Gelatin/MCN-2 | 44 | 2.2 |
| PCL/Gelatin/MCN-3 | 45 | 1.8 |

The effect of PCL degradation on environmental pH in the presence of different nanostructures was evaluated for hybrid scaffolds. The pH changes and mass loss of different scaffolds over 42 days under physiological conditions are presented in FIGS. 5C and 5D, respectively. FIG. 5C presents the % weight loss over time of the tested scaffolds, showing PCL degradation. This data is also represented in Table 3 below, while it is recognized that the corresponding graph includes error bars or ranges for the observed values.

TABLE 3
Mass loss of sample scaffolds (as remaining
percentage of initial mass)

| | | | PCL/ | PCL/ | PCL/ | PCL/ |
|---|---|---|---|---|---|---|
| Time | | PCL/ | Gelatin/ | Gelatin/ | Gelatin/ | Gelatin/ |
| (day) | PCL | Gelatin | Mg(OH)₂ | MCN-1 | MCN-2 | MCN-3 |
| 0 | 100 | 100 | 100 | 100 | 100 | 100 |
| 2 | 81 | 84 | 86 | 88 | 89 | 87 |
| 5 | 78 | 82 | 85 | 86 | 88 | 86 |
| 7 | 76 | 80 | 83 | 83 | 85 | 84 |
| 14 | 51 | 63 | 71 | 74 | 72 | 73 |
| 21 | 23 | 41 | 59 | 66 | 61 | 64 |
| 28 | 14 | 28 | 33 | 47 | 48 | 46 |
| 42 | 2 | 18 | 23 | 30 | 29 | 31 |

FIG. 5D presents the measured changes in pH over the same time. This data is also represented in Table 4 below, while it is recognized that the corresponding graph includes error bars or ranges for the observed values which are within the scope of the present disclosure.

TABLE 4
pH changes during the degradation process of sample scaffolds

| | | | PCL/ | PCL/ | PCL/ | PCL/ |
|---|---|---|---|---|---|---|
| Time | | PCL/ | Gelatin/ | Gelatin/ | Gelatin/ | Gelatin/ |
| (day) | PCL | Gelatin | Mg(OH)2 | MCN-1 | MCN-2 | MCN-3 |
| 0 | 7.4 | 7.4 | 7.4 | 7.4 | 7.4 | 7.4 |
| 2 | 6.9 | 7 | 7.5 | 7.5 | 7.3 | 7.6 |
| 5 | 6.8 | 6.9 | 7.7 | 7.6 | 7.5 | 7.4 |
| 7 | 6.5 | 6.6 | 7.8 | 7.4 | 7.4 | 7.2 |
| 14 | 4.5 | 5.9 | 7.9 | 7.2 | 7.1 | 6.8 |
| 21 | 2.8 | 4.7 | 6.4 | 6.9 | 6.8 | 6.6 |
| 28 | 2.3 | 3.1 | 5.2 | 6.3 | 6 | 5.9 |
| 42 | 2.4 | 2.5 | 5.1 | 6.1 | 5.8 | 5.4 |

Due to acidic byproducts during PCL degradation, the pH value is expected to decrease from the initial value of 7.3 in the media. At the end of the test period (42 days), the PCL scaffold had degraded by 97% and the media pH fallen to an acidic 2.4. While the PCL/Gelatin scaffold degraded by 80% a similar media pH was observed at the end of the test period. The slight difference detected in the pH curve between the PCL and PCL/Gelatin samples might be related to the protein buffer system of gelatin. In the protein buffer system, the histidine content in the protein structure could bind to small amounts of acidic residue. To the contrary, the presence of Mg(OH)₂ buffers the pH, with the presence of MCN further buffering compared to Mg(OH)₂ alone. These results indicate the substantial buffering potential of MCN to neutralize PCL byproducts. The endpoint values in the pH curve for the MCN scaffolds ranged between 5.6 to 6.4 or between 5.6 to 6.4 (+/−20%). Moreover, maintaining the relative neutrality of the pH significantly impacts the PCL degradation profile. PCL degradation may accelerate in pH values below 3 or above 11. Therefore, control of the pH value by the release of magnesium and particularly to above pH 3 or above about 5.6 can be used to regulate scaffold degradation rate, and rate of production of the acidic byproducts. Gentle and/or prolonged degradation of the scaffold might provide a more appropriate condition for cellular activities and tissue regeneration.

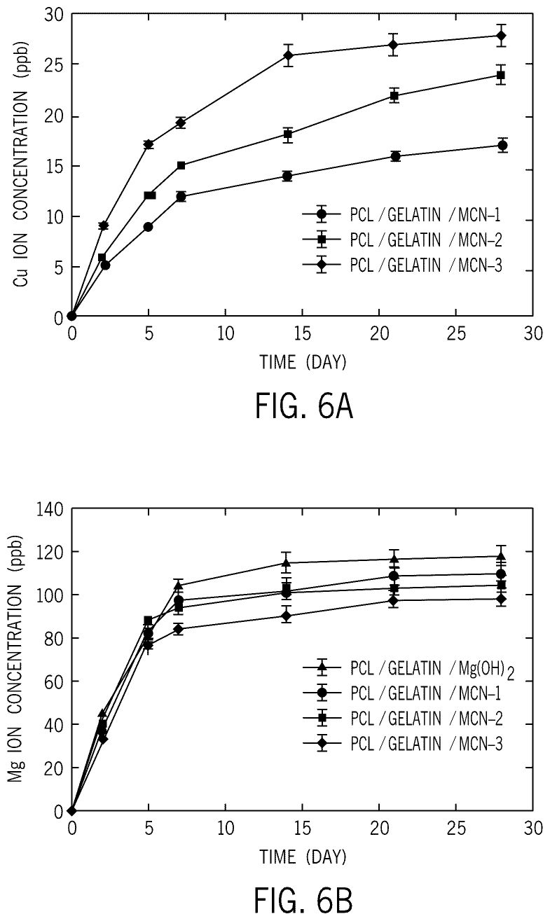

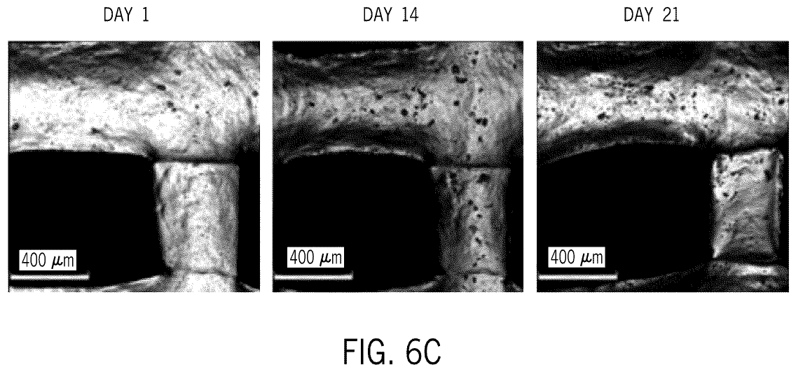

FIGS. 6A-D present data regarding the release of copper and magnesium ions from the disclosed scaffolds. Data values are provided with bars showing ranges for such values. The cumulative release of copper ions from the scaffold over a 28 day test period is shown in FIG. 6A. The MCN-3 scaffold, with the highest concentration of copper, exhibited the highest copper release rate. After 28 days, the MCN-1 scaffold released 16.98 ppb copper ions, the MCN-2 scaffold released 24.32 ppb copper ions, and the MCN-3 scaffold released 27.64 ppb copper ions. The cumulative release of magnesium ions from the scaffold over the 28 day test period is shown in FIG. 6B. This result shows that while all scaffolds exhibited similar release profiles for magnesium ions, the release of copper ions increased over time. FIG. 6C shows SEM images of the degradation of a PCL/gelatin/MCN-2 scaffold. The release profiles of Cu and Mg ions indicates that MCN nanostructures were well incorporated in the gelatin and Cu and Mg ions may be released with the degradation of the gelatin.

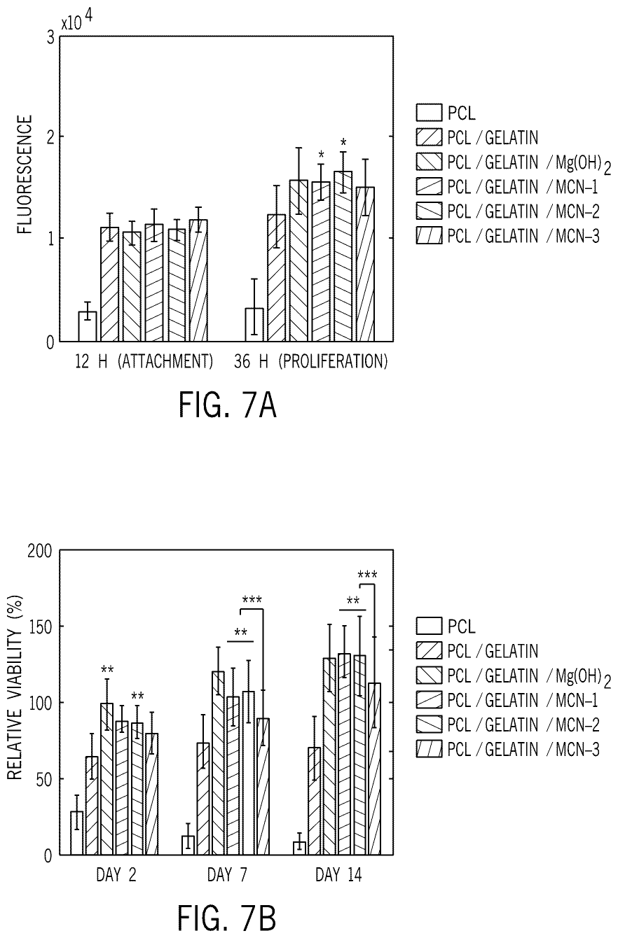

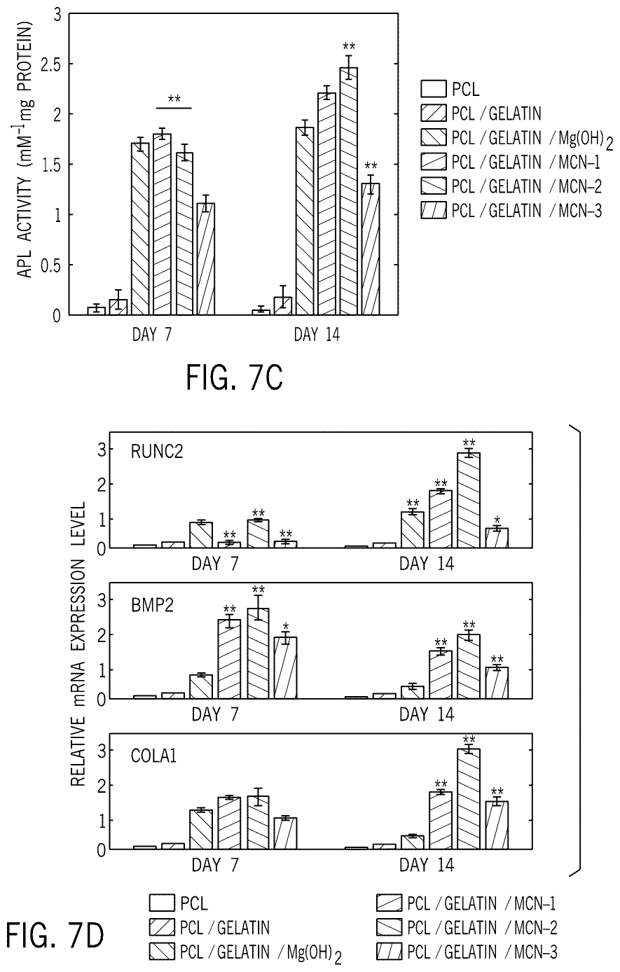

New hybrid nanoparticles, MCNs, composed of copper and magnesium hydroxide were synthesized and the amount of their elements was optimized in a comprehensive in vitro analysis. 3D printed PCL/Gelatin/MCN hybrid scaffold were designed and fabricated at the Mg(OH)₂, MCN-1, MCN-2, and MCN-3 formulations. The effect of the difference scaffolds for osteogenesis of MC3T3-E1 cells proliferation was tested. The results of these tests are reported in FIGS. 7A-D. Data values are provided with bars showing ranges for such values. The viability of the MC3T3-E1 cells at 12 and 36 hours after the initial sell seeding was evaluated using a PB assay, the results of which are reported in the graph of FIG. 7A. All scaffolds except for the isolated PCL scaffold showed a significant increase in cell proliferation at 12 and 36 hours. The graph of FIG. 7B presents the MC3T3-E1 cell relative viability over 2, 7 and 14 day time intervals. The scaffolds with the Mg(OH)₂ or MCN exhibited notably higher viability compared to the PCL or PCL/gelatin scaffold.

Alkaline phosphatase (ALP) activity is an indicator of osteoblast cells and the formation of new bone. The ALP activity of MC3T3-E1 cells on the various scaffolds was tested and the results reported in the graph of FIG. 7C. All Mg(OH)₂ or MCN scaffolds presented significantly more activity than the PCL or PCL/gelatin scaffolds. After 7 days the MCN-1 scaffold produced the highest ALP activity, while the MCN-2 scaffold produced the highest ALP activity after 14 days. Relative mRNA expression levels of the Cola1, Bmp2, and Runx2 genes in MC3T3-E1 cells were tested at 7 and 14 days and the results presented in the graph of 7D. While, particularly by day 14, all of the scaffolds with the Mg(OH)₂ or MCN compositions resulted in increased gene expression, the MCN-2 scaffold presented the highest gene expression level across the tests in this graph. In osteogenic differentiation, the ALP activity is an important early marker.

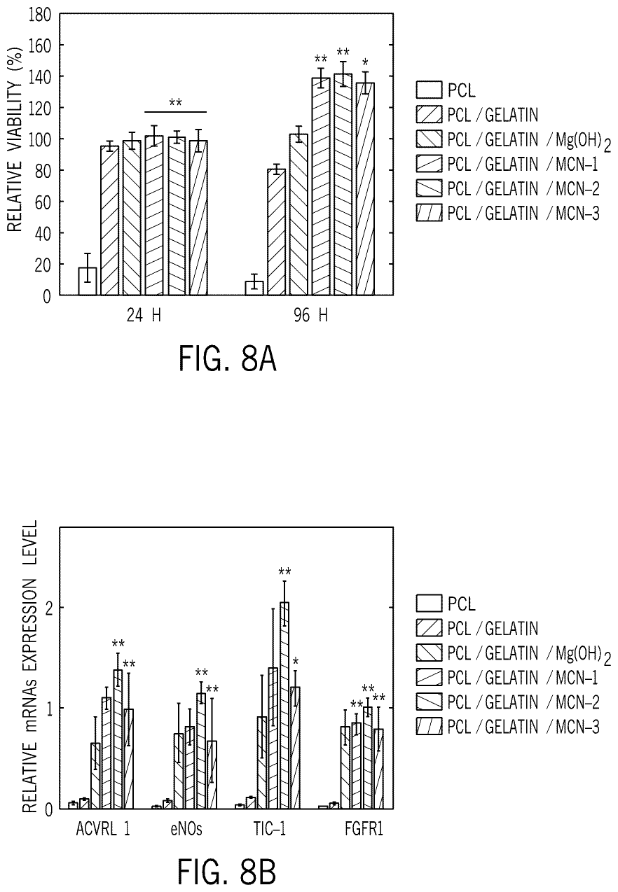

Angiogenesis of the scaffolds were next evaluated. The various disclosed scaffold constructions were again tested for the propagation of HUVECs cells. The results of these tests are shown at FIGS. 8A and 8B. Data values are provided with bars showing ranges for such values. The viability of HUVECs cells at 24 and 96 hours after the initial cell seeding was evaluated using a PB assay and the relative viability reported in the graph of FIG. 8A. After 24 hours, all scaffolds except the PCL only control produced an increase in relative viability. However, after 96 hours, the MCN scaffolds produced significantly higher relative viability of HUVECs cells, with MCN-2 producing the highest viability %. The graph of FIG. 8B presents the relative mRNA expression of the ACVRL1, eNOs, TIE-1, and FGFR1 genes. Like with the osteogenesis evaluation, all of the scaffolds with the Mg(OH)₂ or MCN compositions resulted in increased gene expression, however, MCN-2 produced the greatest gene expression levels across all of the genes investigated.

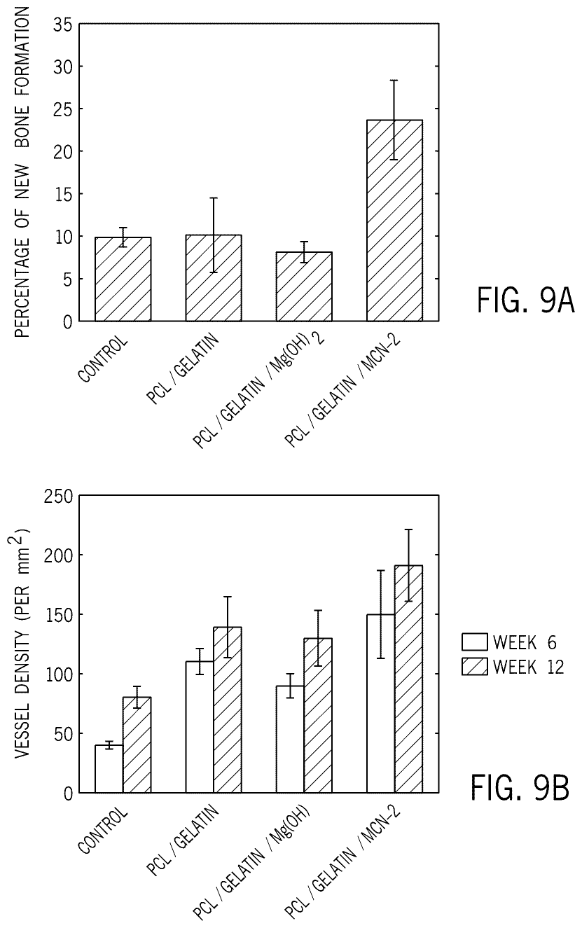

As a result of the tests above, the MCN scaffolds as disclosed herein exhibit improved osteogenesis and angiogenesis with the co-delivery of Mg and Cu ions. Moreover, the relative composition mix provided by the MCN-2 scaffold, produced maximal results across the tests. Based upon those tests, the MCN-2 scaffold was selected for in vivo testing. Scaffolds of PCL/gelatin, PCL/gelatin/Mg(OH)₂, and PCL/gelatin/MCN-2 were tested against a control of no scaffold in laboratory rats after 6 weeks and 12 weeks. Compared to the other scaffold samples in the graph of 9A, the new bone formation area as a percentage of the total defect area was significantly more for the MCN-2, while there was no significant difference between bone growth with the other three tests. All scaffolds saw more new vessels and vessel density (FIG. 9B) compared to the control, but the MCN-2 scaffold saw significantly more vessel density, with vessel density at 6 weeks exceeding the vessel densities of any of the other scaffolds at 12 weeks of growth. Statistical analysis of the above results showed that the percentage of newly formed bone in the defect in the MCN-2 scaffold was significantly higher than the other three tested groups (FIGS. 9A, 9B).

Immunostaining of defect area for CD31 biomarker in the cytoplasmic membrane of endothelial cells represents numerous strongly expressed CD313 vascular structures in the MCN-2 test, with fewer vessel in the other scaffold groups tested. The MCN-2 scaffold enhanced the most newly formed vascular structures that highly expressed CD31 and showed the most new bone area.

The in vitro and in vivo studies show that this multiple-function scaffold could simultaneously promote angiogenesis and osteogenesis by releasing Cu and Mg ions. Moreover, no inflammatory response was observed in the fabricated scaffold due to the neutralization of the acidic byproduct of PCL by Mg ions. The designed scaffold with effective angiogenesis and osteogenesis properties shows promising potential for bone regeneration especially in the defects such as critical-sized ones, in which the effective angiogenesis plays an important role in tissue regeneration. The designed scaffold with effective angiogenesis and osteogenesis properties shows promising potential for bone regeneration especially in the defects such as critical-sized ones, in which the effective angiogenesis plays an important role in tissue regeneration. Scaffolds as disclosed herein are thus suitable for treatment of tissue engineering defects, e.g., mandible, soft tissue, bone, and soft tissue where major challenge is vascularization, especially for people with cancer (removed tumor) as radiotherapy dries out area and causes vascularization issues such that patients cannot eat or speak.

The following are a listing of non-limiting examples of the scaffold for bone tissue engineering as described above.

A first example of a scaffold for bone-tissue engineering includes a mesh and a coating on the mesh. The mesh includes a plurality of offset layers of filaments of polyester. The coating covers the mesh and includes magnesium hydroxide/copper oxide nanoparticles suspended in gelatin.

In a second example from the first example, polyester of the mesh may be polycaprolactone (PCL).

In a third example from any previous example, the magnesium-copper nanoparticles may have a concentration of 0-5 g, 1-4 g, 1.2-3.6 g, 1.6-2.4 g, 2-3 g, or about 2.4 g copper acetate per 75 mL of 2M magnesium chloride solution.

In a fourth example from any previous example, the mesh may have a porosity of up to 1 mm×1 mm or may have a porosity of 400 μm×400 μm.

In a fifth example from any previous example, the coating may be up to 2 μm thick, or may be between 1 μm-2 μm thick.

In a sixth example from any previous example, the copper oxide includes at least copper(II) oxide.

In a seventh example from any previous example, the copper oxide may include both copper(I) oxide and copper(II) oxide. The ratio of copper(I) oxide to copper(II) oxide may be less than 0.42 or may be less than 0.32.

In an eighth example from any previous example, the magnesium hydroxide/copper oxide nanoparticles may have an average particle size of 150 nm or less, optionally the average particle size may be about 120 nm.

In a ninth example from any previous example, the magnesium hydroxide/copper oxide nanoparticles may be present in the gelatin at a concentration of 40 mg-60 mg of magnesium hydroxide/copper oxide nanoparticles to 1 mL gelatin.

In a tenth example from any previous example, the scaffold may have an average pore size of between 351 μm-505 μm.

In an eleventh example from any of previous example, the scaffold may be configured to maintain an invivo localized pH greater than 3.

An example of a method of constructing a scaffold for bone-tissue engineering of any of the previous examples includes 3D printing a mesh including a plurality of offset layers of filaments of polyester. Magnesium hydroxide/copper oxide nanoparticles are prepared by combining 2M solution of magnesium chloride with copper acetate. The magnesium hydroxide/copper oxide nanoparticles are combined with a gelatin solution at a concentration of 40 mg-60 mg or between 45 mg-55 mg of magnesium hydroxide/copper oxide nanoparticles to 1 mL gelatin. Amino-functional groups introduced to the mesh by immersing the mesh in a solution of 1,6 hexanediamine in isopropanol. The mesh is soaked in the combined magnesium hydroxide/copper oxide nanoparticle and gelatin solution.

In the above description, certain terms have been used for brevity, clarity, and understanding. No unnecessary limitations are to be inferred therefrom beyond the requirement of the prior art because such terms are used for descriptive purposes and are intended to be broadly construed. The different systems and method steps described herein may be used alone or in combination with other systems and methods. It is to be expected that various equivalents, alternatives, and modifications are possible within the scope of the appended claims.

The functional block diagrams, operational sequences, and flow diagrams provided in the Figures are representative of exemplary architectures, environments, and methodologies for performing novel aspects of the disclosure. While, for purposes of simplicity of explanation, the methodologies included herein may be in the form of a functional diagram, operational sequence, or flow diagram, and may be described as a series of acts, it is to be understood and appreciated that the methodologies are not limited by the order of acts, as some acts may, in accordance therewith, occur in a different order and/or concurrently with other acts from that shown and described herein. For example, those skilled in the art will understand and appreciate that a methodology can alternatively be represented as a series of interrelated states or events, such as in a state diagram. Moreover, not all acts illustrated in a methodology may be required for a novel implementation.

This written description uses examples to disclose the invention, including the best mode, and also to enable any person skilled in the art to make and use the invention. The patentable scope of the invention is defined by the claims and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A scaffold for bone-tissue engineering, the scaffold comprising:
a mesh comprising a plurality of offset layers of filaments of polyester; and
a coating comprising magnesium hydroxide/copper oxide nanoparticles suspended in gelatin, the coating covering the mesh.

2. The scaffold of claim 1, wherein the polyester is polycaprolactone (PCL).

3. The scaffold of claim 1, wherein the magnesium hydroxide/copper oxide nanoparticles have a concentration of 0-5 g copper acetate per 75 mL of 2M magnesium chloride solution.

4. The scaffold of claim 1, wherein the magnesium hydroxide/copper oxide nanoparticles have a concentration of 1-4 g copper acetate per 75 mL of 2M magnesium chloride solution.

5. The scaffold of claim 1, wherein the magnesium hydroxide/copper oxide nanoparticles have a concentration of 1.2-3.6 g copper acetate per 75 mL of 2M magnesium chloride solution.

6. The scaffold of claim 1, wherein the magnesium hydroxide/copper oxide nanoparticles have a concentration of 1.6-2.4 g copper acetate per 75 mL of 2M magnesium chloride solution.

7. The scaffold of claim 1, wherein the mesh has a porosity up to 1 mm×1 mm.

8. The scaffold of claim 1, wherein the mesh has a porosity of 400 μm×400 μm.

9. The scaffold of any of claim 1, wherein the coating is up to 2 μm thick.

10. The scaffold of any of claim 1, wherein the coating is between 1 μm-2 μm thick.

11. The scaffold of claim 1, wherein the copper oxide of the magnesium hydroxide/copper oxide nanoparticles comprises at least copper(II) oxide.

12. The scaffold of claim 11, wherein the copper oxide of the magnesium hydroxide/copper oxide nanoparticles comprises both copper(I) oxide and copper(II) oxide.

13. The scaffold of claim 12, wherein a ratio of copper(I) oxide to copper(II) oxide is less than 0.42.

14. The scaffold of claim 13, wherein a ratio of copper(I) oxide to copper(II) oxide is less than 0.32.

15. The scaffold of claim 1, wherein the magnesium hydroxide/copper oxide nanoparticles have an average particle size of about 150 nm or less.

16. The scaffold of claim 15, wherein the magnesium hydroxide/copper oxide nanoparticles have an average particle size of about 120 nm.

17. The scaffold of claim 1, wherein the magnesium hydroxide/copper oxide nanoparticles are present in the gelatin at a concentration of 40 mg-60 mg of the magnesium hydroxide/copper oxide nanoparticles to 1 mL of the gelatin.

18. The scaffold of claim 1, wherein the scaffold has an average pore size of between 351 μm-505 μm.

19. The scaffold of claim 1, wherein the scaffold is configured to maintain an invivo localized pH greater than 3.

20. A method of constructing a scaffold for bone-tissue engineering according to claim 1, the method comprising:
3D printing a mesh comprising a plurality of offset layers of filaments of polyester;
preparing magnesium hydroxide/copper oxide nanoparticles by combining 2M solution of magnesium chloride with copper acetate;
combining the magnesium hydroxide/copper oxide nanoparticles with a gelatin solution at a concentration of 40 mg-60 mg of the magnesium hydroxide/copper oxide nanoparticles to 1 mL of the gelatin;
introducing amino-functional groups to the mesh by immersing the mesh in a solution of 1,6 hexanediamine in isopropanol; and
soaking the mesh in the combined magnesium hydroxide/copper oxide nanoparticles and gelatin solution.
